(19) United States
(12) Patent Application Publication
(10) Pub. No.: US 2004/0147020 A1
(43) Pub. Date: Jul. 29, 2004

(54) Dopamine neurons from human embryonic stem cells

(75) Inventors: Curt R. Freed, Denver, CO (US); Kimberley A. Buytaert-Hoefen, Lakewood, CO (US)

Correspondence Address: Christine A. Lekutis, MEDLEN & CARROLL, LLP, Suite 350, 101 Howard Street, San Francisco, CA, 94105, US

(73) Assignee: The Regents of the University of Colorado, a body corporate

(21) Appl. No.: 10/699,302

(22) Filed: Oct. 30, 2003

Related U.S. Application Data
(60) Provisional application No. 60/423,348, filed on Nov. 1, 2002.

(51) Int. Cl. C12N 5/08

(52) U.S. Cl. 435/368

(57) ABSTRACT

The present invention relates to methods and compositions for differentiating human embryonic stem cells into dopamine producing neurons. The methods and compositions of the present invention are suitable for use in the treatment of neurological disorders.

### FIELD OF THE INVENTION

The present invention relates to methods and compositions for differentiating human embryonic stem cells into dopamine producing neurons. The methods and compositions of the present invention are suitable for use in the treatment of neurological disorders such as Parkinson's disease.

### BACKGROUND OF THE INVENTION

Parkinson's disease is a chronic, progressive, hypokinetic disorder characterized by impaired voluntary movement. Symptoms of Parkinson's disease include bradykinesia, tremor, rigidity, and/or atypical gait (See, Dale and Federman (eds.), WebMD Scientific American Medicine, NY: WebMD Corporation, Chapter 11, Section 15, pp.1-21, 2001; Lang and Lozano, N Engl J Med, 339:1044, 1998; and Lang and Lozano, N Engl J Med, 339:1130, 1998). Parkinson's disease occurs as a result of the death of dopamine-producing neurons in the substantia nigra of the midbrain, with symptoms developing when greater than 50% of the midbrain dopamine neurons are lost.

Parkinson's disease afflicts more than one million persons in the United States alone (Lang and Lozano, supra, 1998), with approximately 50,000 new cases diagnosed each year. It is generally a disease of late middle age, with typical onset occurring at about age 60; although about five percent of patients have early-onset disease and are younger than 40 when symptoms begin.

Historically, efforts to treat Parkinson's disease have used dopaminergic medications that either boost dopamine levels in the brain or mimic the effects of dopamine on target cells. The single most effective therapy for Parkinson's disease is the amino acid levodopa or L-Dopa (Cotzias et al., N Engl J Med, 276:374-379, 1967), which is converted into dopamine in the brain after ingestion. Unfortunately, long-term use of levodopa is associated with debilitating complications in most patients including motor fluctuations, dyskinesia (e.g., abnormal, uncontrollable movements), and/or mental impairments such as confusion or hallucinations.

Surgical treatments are considered for patients with advanced disease or who have not responded adequately to medications. Accepted surgical treatments involve either the creation of small, precise lesions or the implantation of stimulating electrodes in specific brain regions that appear to be overactive in Parkinson's disease. Thalamotomy involves electrocoagulation of portions of the thalamus to interrupt nervous transmission through the thalamus for relief of tremors that do not respond to medications. Pallidotomy employs an electrode to destroy cells of the globus pallidus to relieve dyskinesias caused by levodopa therapy, as well as to provide some relief from rigidity and tremor. Additionally, subthalamic nucleus stimulation may also be effective in relieving Parkinson's disease symptoms and reducing levodopa-induced dyskinesias.

More recently, implantation of fetal dopaminergic tissue has been utilized as a means of restoring dopamine levels in the brains of patients with Parkinson's disease (See, e.g., Freed et al., Arch Neurol, 47:505-512, 1990; and Lindvall et al., Science, 247:574-577, 1990). However, there are two major problems associated with fetal dopamine neuron transplants. First is the difficulty in recovering sufficient quantities of brain tissue from elective abortions. Second is the poor survival of the transplanted dopamine neurons. In fact, 90 to 95% of the transplanted neurons fail to survive, with most dying by programmed cell death (Kordower et al., N Engl J Med, 332:1118-1124, 1995; Freed et al., N Engl J Med, 344:710-719, 2001; and Clarkson et al., Exp Neurol, 168:183-191, 2001). Thus, what is needed in the art are methods and compositions for the expansion of dopamine neurons suitable for transplantation.

### SUMMARY OF THE INVENTION

The present invention relates to methods and compositions for differentiating human embryonic stem cells into dopamine producing neurons. The methods and compositions of the present invention are suitable for use in the treatment of neurological disorders such as Parkinson's disease.

In particular, the present invention provides methods for producing tyrosine hydroxylase-positive neurons, comprising the steps of: providing an embryonic stem cell line; and contacting the embryonic stem cell line with the Otx2 transcription factor, FGF8 protein, and Sonic hedgehog protein, under conditions suitable for producing tyrosine hydroxylase-positive neurons. Some embodiments, further comprise providing a N-2 growth supplement, and contacting the embryonic stem cell line with the N-2 growth supplement. Other embodiments, further comprise providing feeder cells and contacting the embryonic stem cell line with the feeder cells. Some preferred embodiments, further comprise providing a conditioned medium and contacting the embryonic stem cell line with the conditioned medium. Additionally the present invention provides embodiments in which the Otx2 transcription factor is provided by infecting the embryonic stem cell line with a recombinant adenovirus comprising an Otx2 expression cassette. In particularly preferred embodiments, the embryonic stem cell line is a human embryonic stem cell line. In some embodiments, the FGF8 and the Sonic hedgehog proteins are purified proteins each provided at a concentration range of 1 ng/ml to 10 μg/ml, while in other embodiments, the FGF-8 and the Sonic hedgehog proteins are purified proteins each provided at a concentration of 10 ng/ml to 1 μg/ml. In some preferred embodiments, the feeder cells comprises embryonic striatum cells, while in other embodiments, the conditioned medium comprises medium conditioned by embryonic striatum cells.

The present invention also provides cell cultures produced by a method comprising contacting an embryonic stem cell line with an FGF8 protein and a Sonic hedgehog protein, under conditions suitable for producing neurons. In some embodiments, at least 50% of the neurons express tyrosine hydroxylase. In other embodiments, at least 50% of the neurons express the dopamine transporter. In preferred embodiments, the neurons are suitable graft material for human transplantation, while in particularly preferred embodiments, the human transplantation comprises treatment of a subject displaying symptoms of Parkinson's disease.

Moreover, the present invention provides methods of alleviating Parkinson's disease symptoms in a patient with Parkinson's disease comprising: providing a cell culture produced by a method comprising contacting a human embryonic stem cell line with an Otx2-expressing gene, an FGF8 protein, and a Sonic hedgehog protein, under conditions suitable for producing tyrosine hydroxylase-positive neurons; and administering the cultured cells comprising tyrosine hydroxylase-positive neurons to the putamen of a patient with Parkinson's disease, under conditions suitable for alleviating Parkinson's disease symptoms. In some embodiments, alleviating the Parkinson's disease symptoms is assessed by the use of the Unified Parkinson's Disease Rating Scale, alleviating the Parkinson's disease symptoms is assessed by the use of the Schwab and England Scale, or alleviating the Parkinson's disease symptoms is assessed by the use of the Core Assessment Program for Intracerebral Transplantation. The patient has advanced Parkinson's disease in some embodiments of the present invention.

Additionally, the present invention provides methods for producing tyrosine hydroxylase-positive neurons, comprising the steps of: providing a human embryonic stem cell line; and contacting the embryonic stem cell line with at least one soluble molecule expressed by fetal striatal cells, under conditions suitable for producing tyrosine hydroxylase-positive neurons. In some embodiments, the fetal striatal cells are astrocytes. In preferred embodiments, the at least one soluble molecule comprises glial-derived neurotrophic factor. The present invention provides embodiments in which the fetal striatal cells are cocultured with the human embryonic stem cell line. In some preferred embodiments, the fetal striatal cells are separated from the human embryonic stem cell line by a semipermeable membrane, in a subset of these embodiments, the semipermeable membrane has a pore size of 0.4 microns or less. Also provided are embodiments in which the at least one soluble molecule is provided by conditioned medium from fetal striatal cell cultures. In preferred embodiments, the conditioned medium is free of the fetal striatal cells. Other embodiments of the present invention further comprise contacting the human embryonic stem cell line with stromal cells. Stromal cells or other supportive cells may be genetically modified to express the wnt-1 gene to enhance differentiation to tyrosine hydroxylase positive neurons and dopamine transporter-positive neurons. Some preferred embodiments further comprise step c, enriching the tyrosine hydroxylase positive neurons, while in related embodiments, the tyrosine hydroxylase positive neurons are enriched by selecting colonies with a circumference greater than 4 mm.

Furthermore, the present invention provides cell cultures produced by a method comprising contacting a human embryonic stem cell line with at least one soluble molecule expressed by fetal striatal cells, under conditions suitable for producing tyrosine hydroxylase-positive neurons. In preferred embodiments, the at least one soluble molecule comprises glial-derived neurotrophic factor. In some preferred embodiments, the method further comprises enriching the tyrosine hydroxylase positive neurons. In other embodiments, the conditions are further suitable for producing dopamine transporter-positive neurons. In preferred embodiments, the neurons are suitable graft material for human transplantation, while in particularly preferred embodiments, the human transplantation comprises treatment of a subject displaying symptoms of Parkinson's disease.

The present invention also provides methods of alleviating Parkinson's disease symptoms in a patient with Parkinson's disease comprising: providing a cell culture produced by a method comprising contacting a human embryonic stem cell line with at least one soluble molecule expressed by fetal striatal cells, under conditions suitable for producing tyrosine hydroxylase-positive neurons; and administering the cultured cells comprising tyrosine hydroxylase-positive neurons to the putamen of a patient with Parkinson's disease, under conditions suitable for alleviating Parkinson's disease symptoms. In preferred embodiments, the at least one soluble molecule comprises glial-derived neurotrophic factor. In some embodiments, the alleviating Parkinson's disease symptoms is assessed by a technique chosen from but not limited to the Unified Parkinson's Disease Rating Scale, the Schwab and England Scale, and the Core Assessment Program for Intracerebral Transplantation. Also provided are embodiments in which the patient has advanced Parkinson's disease.

In further embodiments, the present invention provides a composition comprising at least one human embryonic stem cell and medium comprising glial-derived neurotrophic factor. In some preferred embodiments, the glial-derived neurotrophic factor is a recombinant protein. In additional preferred embodiments, the medium further comprises at least one soluble molecule expressed by stromal cells, which in particularly preferred embodiments is a recombinant protein.

### DESCRIPTION OF THE FIGURES

FIG. 1 shows results obtained from a RT-PCR analysis of murine dopamine neurons differentiated in vitro using the method described in the first paragraph of Example 3.

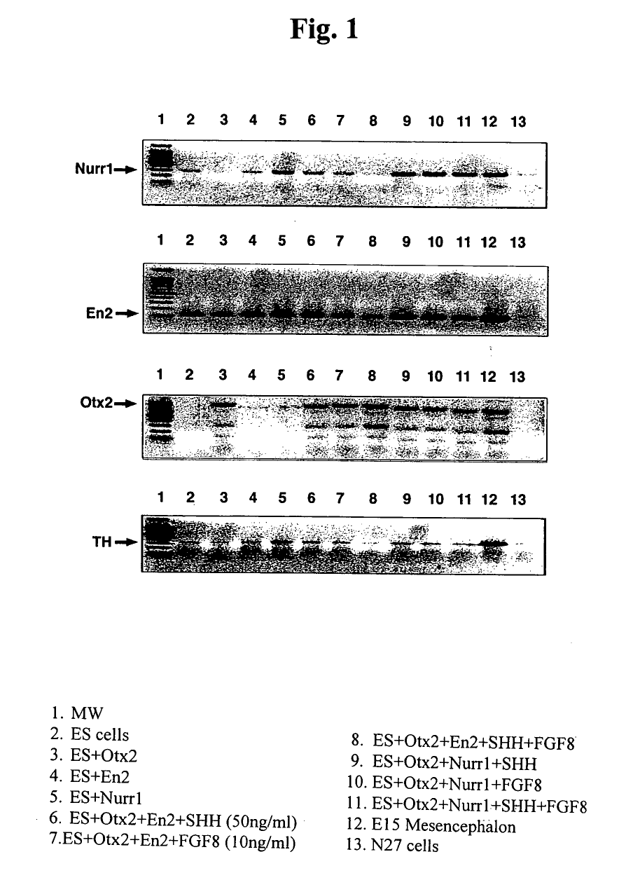

FIG. 2 shows results obtained from a RT-PCR analysis of murine dopamine neurons differentiated in vitro using the alternative method described in the second paragraph of Example 3. Tissue culture methods employing Ad-Otx2, FGF8 and SHH, induced TH and DAT mRNA expression appreciably above that observed in the negative control undifferentiated ES cells and in the ES cells infected with Ad-GFP.

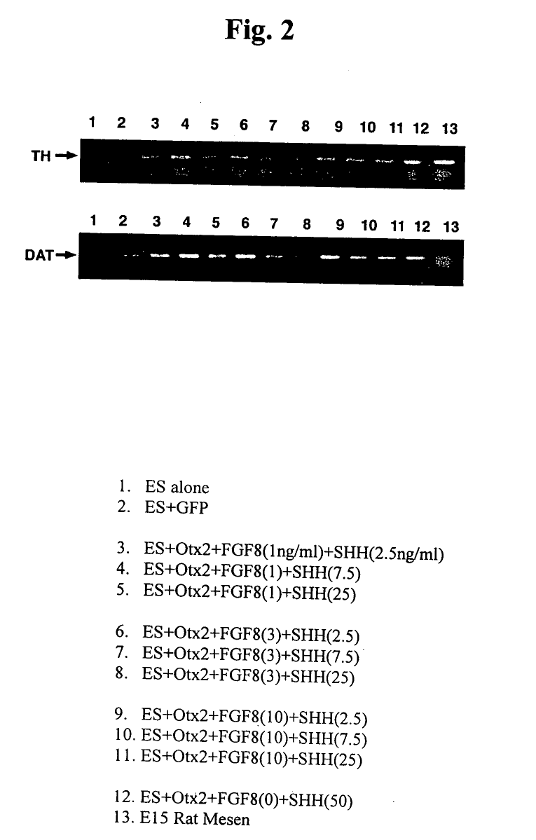

FIG. 3 shows results obtained from a western blot analysis of murine dopamine neurons differentiated in vitro using the alternative method described in the second paragraph of Example 3.

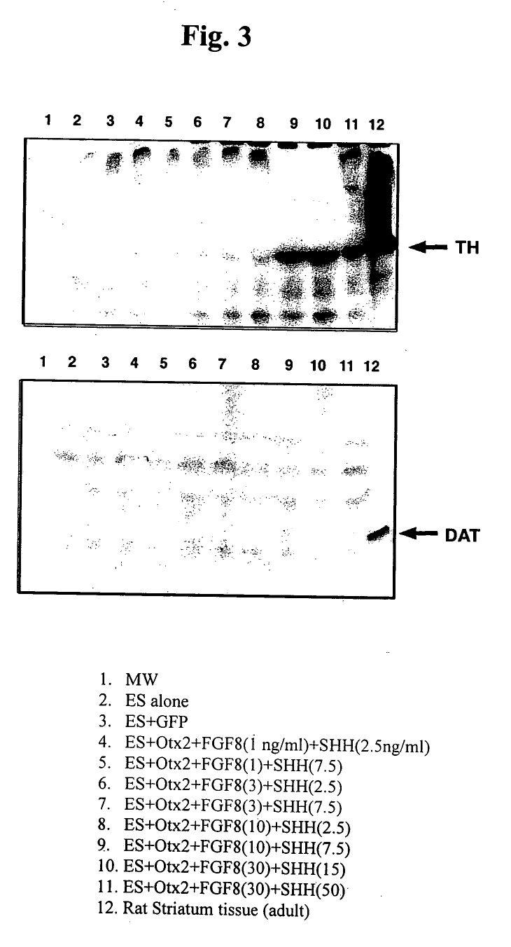

FIG. 4, panels A and B show images of mouse ES cells differentiated for 2 weeks on PA6 monolayers in the presence of fetal rat striatum. Panel C provides a graph of colony circumference versus numbers of TH positive cells obtained upon culturing the ES cells in the presence of PA6 cells and fetal rat striatum fragments.

FIG. 5, panels A and B show images of human ES cells cultured for 2 weeks on PA6 monolayers in the presence or absence of fetal rat striatum. Panel C provides a graph of colony circumference versus numbers of TH positive cells obtained upon culturing the ES cells on PA6 monolayers in the presence or absence of fetal rat striatum fragments. Panels D and E provide graphs indicating that human ES cell exposure to rat striatum fails to yield larger colonies or greater numbers of TH positive cells, respectively.

FIG. 6, panels A, B, and C show images of human ES cells differentiated for 4 weeks on PA6 monolayers in the presence of fetal human striatum. Panel D provides a graph of colony circumference versus numbers of TH positive cells obtained upon culturing the ES cells on PA6 monolayers in the presence or absence of fetal human striatum fragments. Panels E and F provide graphs indicating that human ES cell exposure to fetal human striatum yields similar sized colonies, but with greater numbers of TH positive cells.

FIG. 7, panels A, B, and C show images of human ES cells differentiated for 3 weeks on fetal rat striatal astrocytes. Panel D provides a graph of colony circumference versus numbers of TH positive cells obtained upon culturing the ES cells on fetal rat astrocytes obtained from ventral mesencephalon and striatum, respectively. Panels E and F provide graphs indicating that human ES cell exposure to fetal rat striatal astrocytes yields larger colonies, with greater numbers of TH positive cells.

FIG. 8 provides a graph showing the numbers of TH positive cells obtained by culturing human ES cells in the absence or presence of PA6 monolayers and in the presence or absence of the neurotrophin, termed glial cell-derived neurotrophic factor (GDNF).

### DESCRIPTION OF THE INVENTION

The present invention relates to methods and compositions for differentiating human embryonic stem cells into dopamine producing neurons. The methods and compositions of the present invention are suitable for use in the treatment of neurological disorders such as Parkinson's disease.

A number of sources for transplantable dopamine-producing cells have been explored. An ideal source of graft tissue will have two important characteristics. First, the cells would be renewable in vitro, and second, the graft tissue would be a homogenous population. In light of these and other criteria, cultured stem cells have been explored as a promising source of transplantation material.

There are two general types of stem cells, namely, embryonic stem (ES) cells and multipotent stem cells (sometimes called progenitor cells). ES cells are derived from blastocyst stage mammalian embryos, and represent a very early stage of cell specialization. These cells are self-renewing in vitro, and have the ability to yield many, if not all, of the cell types present in a mature individual. In contrast to ES cells, multipotent stem cells represent an intermediate stage of development that lies between ES cells and fully differentiated cells. Multipotent stem cells have limited proliferative capacity and restricted differentiation capacity, and can be derived from embryonic, fetal or adult tissues. One example of multipotent stem cells are stem cells that have been derived from fetal or adult brain tissue (e.g., neural or CNS stem cells).

Neural stem cells retain the ability to form a variety of neuronal cell types. It is widely believed that the identity of a neuron is determined by its position within the developing brain and, as a result of its position, by its exposure to various regulatory proteins, collectively called differentiating factors (See, e.g., Hynes and Rosenthal, Curr Opin Neurobiol, 9:26-36, 1999). Thus, as is described in detail in the experimental examples, by exposing cultured stem cells to the differentiating factors, which normally induce dopaminergic neurons in vivo, dopaminergic neurons have been produced from ES cell in vitro. In particular, some embodiments of the present invention comprise culture conditions utilizing the differentiating factors wnt1, Otx2, FGF-8 and SHH. Also suitable for use with the present invention are the compositions and methods for forming dopaminergic neurons described in U.S. Pat. No. 6,277,820, herein incorporated by reference in its entirety.

The mammalian Otx2 gene is related to the Drosophila orthodenticle gene (Simeone et al., EMBO J., 12:2735-2747, 1993; and GenBank Accession No. NM021728). The Otx2 gene product is a homeodomain-containing transcription factor, which is required in vivo for mammalian rostral (i.e., forebrain and midbrain) neuroectoderm development during the early stages of CNS specification (Simeone, EMBO J., 17:6790-6798, 1998). In some embodiments of the present invention, the human Otx2 (hOtx2) gene is expressed in ES cells via infection with a recombinant adenovirus (Ad-Otx2). Mammalian homologues of hOtx2 are contemplated to find use in the methods and compositions of the present invention.

Fibroblast growth factor 8 (FGF-8), also known as the androgen-induced growth factor is one member of the fibroblast growth factor family of signaling molecules (GenBank Accession No. AF520763). FGF-8 is locally produced at the mid/hindbrain boundary and in the rostral forebrain, and together with SHH creates induction sites for dopaminergic neurons (Ye et al., Cell, 93:755-766, 1998; and U.S. Pat. No. 6,277,820). In some embodiments of the present invention, ES cell cultures comprise a recombinant murine FGF-8 protein (mFGF-8). Additionally, mammalian homologues (preferably human) of mFGF-8 are contemplated to find use in the methods and compositions of the present invention. In some embodiments of the present invention, ES cell cultures comprise FGF-8b (i.e., b isoform of FGF-8).

The mammalian sonic hedgehog (SHH) gene is related to the Drosophila segment polarity gene known as hedgehog (Marigo et al., Genomics 28:44-51, 1995; and GenBank Accession No. L38518). SHH encodes a secreted protein implicated in regulating the patterning of the developing central nervous system, somite and limb (Echelard et al., Cell 75:1417-1430, 1993; and Krauss et al., Cell 75:1431-1444, 1993; and Riddle et al., Cell 75:1401-1416, 1993). In particular, SHH has been shown to induce dopaminergic neuron differentiation in pre-existing neural tissue in vitro (Hynes et al., Neuron, 15:35-44, 1995; and Wang et al., Nat Med., 1:1184-1188, 1995). In some embodiments of the present invention, ES cell cultures comprise a recombinant amino-terminal fragment (e.g., residues 25-198) of murine SHH. Mammalian homologues (preferably human) of mSHH are contemplated to find use in the methods and compositions of the present invention.

Also provided by the present invention are embodiments comprising feeder cells. In particular, the present invention provides methods and compositions in which ES cells are cultured in the presence of non-ES cells. In preferred embodiments, the non-ES cells comprise at least one different cell type. Suitable feeder cells include but are not limited to embryonic striatum, stromal cells, astrocytes and fibroblasts. In some embodiments, the feeder cells are nerve tissue derived from the embryonic corpus striatum. In other embodiments, the PA6 bone marrow-derived stromal cells (MC3T3-G2/PA6), established from newborn mouse calvaria (Kodama et al., J Cell Physiol, 112:89-95, 1982), are used. In fuirther preferred embodiments, MS-5 stromal cells established by irradiation of adherent cells in bone marrow cultures (Itoh et al., Exp. Hematol., 17:145-153, 1989), are used in place of the PA6 cells.

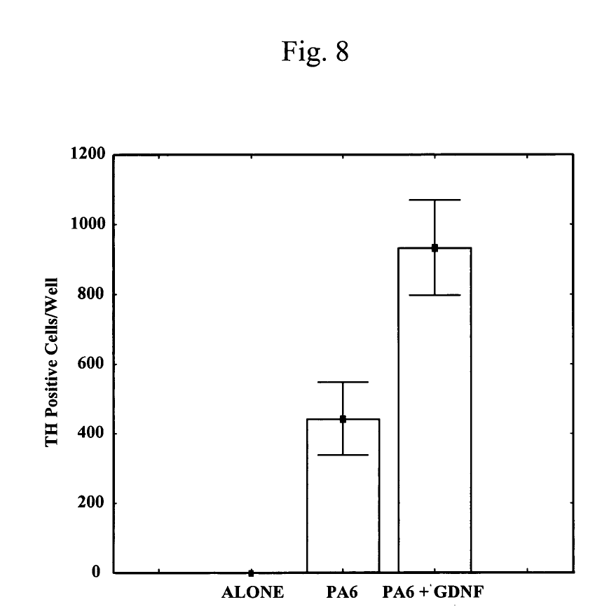

Additionally, in some embodiments, the feeder cells are of human origin, while in other embodiments, the feeder cells are of nonhuman origin (e.g., rodent, porcine, nonhuman primate, etc.). In some preferred embodiments, culture conditions comprising PA6 cells and a neurotrophin such as glial cell-derived neurotrophic factor (GDNF), are provided (See, FIG. 8).

Glial cell-derived neurotrophic factor (GDNF) is a growth factor that enhances the survival and morphological differentiation of dopaminergic neurons and increases their uptake of dopamine (Lin et al., Science, 260:1130-1132, 1993, and GenBank Accession No. L19063). GDNF, which is a ligand for the RET gene, can rescue motor neurons from programmed cell death and death caused by axotomy. GDNF also protects cells from the effect of the neurotoxin MPTP. Mammalian homologues of human GDNF are contemplated to find use in the methods and compositions of the present invention.

As an alternative to the use of feeder cells, embodiments of the present invention comprising conditioned medium are provided. Conditioned medium is obtained by its use in feeder cell cultures. For instance, the medium used to culture feeder cells in vitro is suitable for use in the methods and compositions of the present invention. In particular, conditioned medium from striatum cells is contemplated to contain feeder cell derived molecules (e.g., GNDF) important for the growth and differentiation of neurons. In some embodiments, the conditioned medium is filtered to remove feeder cell contaminants.

Definitions

To facilitate an understanding of the present invention, a number of terms and phrases are defined below:

As used herein, the term “neurodegeneration” refers broadly to a defect involving or relating to the nervous system. As used herein, the terms “neurodegenerative disorder” or “neurodegenerative disease” refer broadly to disorders or diseases that affect the nervous system, including but not limited to Parkinson's disease, Alzheimer's disease, Huntington's disease and amyotrophic lateral sclerosis. As used herein, the term “neurodegeneration is reduced” refers to the improvement in the neurodegenerative condition, such that the degree of neurodegeneration is lessened.

The term “subject suffering from a neurodegenerative disease” as used herein, refers to both humans and animals displaying symptoms normally associated with a disease that affects the nervous system. The term “animals” refers to all non-human animals. Such non-human animals include, but are not limited to, vertebrates such as rodents, non-human primates, ovines, bovines, lagomorphs, porcines, caprines, equines, canines, felines, aves, etc.

As used herein, the term “sample” refers broadly to all types of samples obtained from humans and other animals, including but not limited to, body fluids such as urine, blood, fecal matter, cerebrospinal fluid (CSF), semen, and saliva, as well as solid tissue. These examples are not to be construed as limiting the sample types applicable to the present invention.

The terms “Parkinson's disease,” “Parkinson's” and “PD” refer to a neurological syndrome characterized by a dopamine deficiency, resulting from degenerative, vascular, or inflammatory changes in the basal ganglia of the substantia nigra. This term also refers to a syndrome which resembles Parkinson's disease, but which may or may not be caused by Parkinson's disease, such as Parkinsonian-like side effects caused by certain antipsychotic drugs.

As used herein, the term “early stage of Parkinson's disease” refers broadly to the first stages in Parkinson's disease, wherein a person suffering from the disease exhibits mild symptoms that are not disabling, such as an episodic tremor of a single limb (e.g., the hand), and which affect only one side of the body.

In contrast, the term “advanced stage of Parkinson's disease” refers broadly to a more progressive stage in Parkinson's disease, wherein a person suffering from the disease exhibits symptoms which are typically severe and which may lead to some disability (e.g., tremors encompassing both sides of the body, balance problems, etc.). Symptoms associated with advanced stage Parkinson's disease may vary significantly in individuals, and may take many years to manifest after the initial appearance of the disease.

As used herein, the terms “transplant cells” and “graft material” refer broadly to the component (e.g., tissue or cells) being grafted, implanted or transplanted. As used herein, the term “transplantation” refers to the transfer or grafting of tissues or cells from one part of a subject to another part of the same subject, or to another subject, or the introduction of biocompatible materials into or onto the body. As used herein, a transplanted tissue may comprise a collection of cells of identical or similar composition, or derived from an organism (i.e., a donor), or from an in vitro culture (i.e., a tissue culture system). The term “suitable graft material” refers to tissue with the desired phenotype (e.g., dopamine neuron morphology, and/or expression of the neuronal tyrosine hydroxylase antigen), which is free of deleterious contaminants (e.g., free of bacteria and fungi).

The term “recipient of transplanted cells” as used herein, refers broadly to the subject undergoing transplantation and receiving transplanted cells.

As used herein, the terms “neuron” and “nerve cell” refer to an excitable cell specialized for the transmission of electrical signals. A typical neuron consists of a cell body, an axon, axon terminals, and dendrites. Signals are transmitted in the form of neurotransmitters from the axon of one nerve cell to the dendrite of another nerve cell across a junction known as a synapse,

The term “dopamine neurons” refers broadly to neurons, which produce the neurotransmitter dopamine. The term “dopamine” refers to a catecholamine neurotransmitter and hormone, formed by decarboxylation of dehydroxyphenylalanine (dopa). Dopamine is a precursor of adrenaline and noradrenaline.

As used herein, the terms “tyrosine hydroxylase” and “TH” refer to an enzyme required for the synthesis of the neurotransmitters noradrenaline and dopamine. TH is a commonly accepted marker of dopaminergic neurons. As used herein, a TH+ cell is a cell which immunostains positive using an anti-tyrosine hydroxylase primary antibody.

The terms “dopamine transporter” and “DAT” refer to gene product, which mediates uptake of dopamine into neurons.

As used herein, the term “cell culture” refers to any in vitro culture of cells, including but not limited to continuous cell lines (e.g., with an immortal phenotype), primary cell cultures, and finite cell lines (e.g., non-transformed cells).

The term “coculture” as used herein, refers to the growth of distinct cell types in a combined culture. For instance, in some embodiments, ES cells are placed in culture at a low density with striatal cells and/or stromal cells. When it is desirable to prevent contamination of the differentiated ES cells (e.g., dopamine neurons) with the striatal or stromal feeder cells, the feeder cells may be treated with mitomycin C or irradiation to prevent feeder cell replication. Alternatively, the feeder cells may be separated from the ES cells by a mechanical means such as a semipermeable membrane.

As used herein the term “semipermeable membrane” refers to a membrane, which is freely permeable to water (or other solvents) but relatively impermeable to solutes. In some embodiments, the membrane is merely impermeable to very large molecules (e.g., greater than 100 kD), while in other embodiments, the membrane is merely impermeable to cells (e.g., greater than 2 to 4 microns).

The term “in vitro” refers to an artificial environment and to processes or reactions that occur within an artificial environment. The term “in vivo” refers to the natural environment (e.g., an animal or a cell) and to processes or reactions that occur within a natural environment. The definition of an in vitro versus in vivo system is particular for the system under study. As used herein, an in vitro system refers to studies of cells or processes in an artificial environment, such as in tissue culture vessels and apparatus, whereas study of the same system in an in vivo context refers to the study of cells or processes within an organism, such as a rat or human.

As used herein, the term “primary cell” or “primary culture” refers to a cell or a culture of cells that have been explanted directly from an organism. Primary cultures are neither transformed nor immortal.

The term “tissue culture” as used herein, refers to a collection of techniques for the growth and maintenance of eukaryotic cells in the laboratory. Such techniques may involve tissue culture dishes or other vessels, incubators and sterility containment devices, as known in the art. The term “cell culture” refers to cells maintained in the laboratory.

As used herein, the term “exogenous” is used interchangeably with the term “heterologous” refer to a substance coming from some source other than its native source. For example, the terms “exogenous protein,” or “exogenous cell” refer to a protein or cell from a non-native source, and that have been artificially supplied to a biological system. In contrast, the terms “endogenous protein,” or “endogenous cell” refer to a protein or cell that are native to the biological system, species or individual.

The terms “embryonic stem cell” and “ES cell” refer to cells derived from mammalian embryos or blastocysts, which are self-renewing and have the ability to yield many or all of the cell types present in a mature animal. Human embryonic stem cell lines suitable for use with the methods and compositions of the present invention include but are not limited to those produced by the following institutions: BresaGen, Inc., Athens, Ga.; CyThera, Inc., San Diego, Calif.; ES Cell International, Melbourne, Australia; Geron Corporation, Menlo Park, Calif.; Goteborg University, Goteborg, Sweden; Karolinska Institute, Stockholm, Sweden; Maria Biotech Co. Ltd.—Maria Infertility Hospital Medical Institute, Seoul, Korea; MizMedi Hospital—Seoul National University, Seoul, Korea; National Centre for Biological Sciences/Tata Institute of Fundamental Research, Bangalore, India; Pochon CHA University, Seoul, Korea; Reliance Life Sciences, Mumbai, India; Technion University, Haifa, Israel; University of California, San Francisco, Calif.; and Wisconsin Alumni Research Foundation, Madison, Wis. The human ES cells listed on the Human Embryonic Stem Cell Registry to be created by the National Institutes of Health find use in the methods and compositions of the present invention. However, human ES cells not listed on the NIH registry are also contemplated to find use in embodiments of the present invention (e.g., when it is desirable to prevent ES contamination with nonhuman-derived materials).

As used herein the term “feeder cells” refers to cells used as a growth support in a tissue culture system. In preferred embodiments, the term “feeder cells” refers to for example: striatum cells, stromal cells or astrocytes, although the present invention is not limited to the use of any of these types of cells.

The terms “corpus striatum” and “striatum” refer to gray and white matter consisting of the neostriatum and paleostriatum (globus pallidus), located in front of and lateral to the thalamus in each cerebral hemisphere. The gray substance is made up of the caudate nucleus and the lentiform nucleus (the latter consisting of the globus pallidus and putamen). The white matter is the internal capsule. The term “putamen” refers to the outer layer of gray matter in the lentiform nucleus.

As used herein, the term “stromal cells” refers to connective tissue cells of an organ found in the loose connective tissue. In preferred embodiments, the term refers to cells (e.g., PA6), which possess stromal cell-derived inducing activity (Kawasaki et al., Neuron, 28:31-40, 2000).

The term “astrocytes” as used herein refers to the largest and most numerous neuroglial cells in the brain and spinal cord. Astrocytes are irregularly shaped with many long processes, including those with “end feet” which form the glial (limiting) membrane and directly and indirectly contribute to the blood-brain barrier.

As used herein, the term “embryonic” means undeveloped or related to an embryo. In humans, the term embryo refers to the developing organism from about two weeks after fertilization to the end of seventh or eighth week. As used herein, the term “fetal” refers to in utero development occurring after the embryonic period. In humans, the term “fetus” refers to the developing organism after about seven or eight weeks of pregnancy. In some embodiments of the present invention, the feeder cells may be of fetal or embryonic origin.

The term “gene” refers to a nucleic acid (e.g., DNA) sequence that comprises coding sequences necessary for the production of a polypeptide or precursor (e.g., Otx2). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term “nucleotide sequence” encompasses DNA, cDNA, and RNA sequences. In particular, the term “Otx2 nucleotide sequence” refers to the coding region of the Otx2 gene.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “express” refers to the use of the information in a gene via transcription and/or translation, which ultimately leads to production of a protein and the appearance of phenotype determined by that gene.

The term “Otx2” refers to a homeodomain-containing transcription factor. The human Otx2 gene is disclosed as GenBank Accession No. NM021728, however, the present invention is not limited to the human gene.

The terms “fibroblast growth factor 8” and “FGF-8” refer to the androgen-induced growth factor. The human FGF-8 gene is disclosed as GenBank Accession No. AF520763, however, the present invention is not limited to the human gene. In fact, some embodiments of the present invention utilize recombinant mouse FGF-8 protein.

The terms “SHH” and “sonic hedgehog” refer to a secreted protein, which regulates patterning of the developing central nervous system. The human SHH gene is disclosed as GenBank Accession No. L38518, however, the present invention is not limited to the human gene. In fact, some embodiments of the present invention utilize a recombinant mouse SHH protein fragment.

The terms “GDNF,” “glial cell-derived neurotrophic factor,” “ATF1,” and “astrocyte-derived trophic factor 1” refer to a growth factor that enhances survival of midbrain dopaminergic neurons. The human GDNF gene is disclosed as GenBank Accession No. L19063, however, the present invention is not limited to the human gene. Some embodiments of the present invention utilize recombinant GDNF protein or a fragment of the GDNF protein (e.g., isoform or truncated versions of GDNF).

As used herein, the term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

As used herein, the term “N-2 supplement” refers to a chemically-defined media supplement formulated for culturing neurons (Bottenstein, in Cell Culture in the Neurosciences, Bottenstein and Harvey (eds.), Plenum Press: NY, p. 3, 1985). N-2 supplement contains insulin, transferrin, progesterone, putrescine and selenite, and is commercially-available from Invitrogen The terms “conditioned medium,” “CM” and “cell-free culture supernatant” refer to media in which cells have been cultivated already for a period of time. The medium is generally obtained by sterile filtration of used culture media and is added to fresh culture media for some portion of the final volume. Conditioned media are used for the cultivation of particularly fastidious cells and cell lines (e.g., ES cells) because CM contains mediator substances such as growth factors and cytokines, which promote the growth of new cells.

The term “soluble molecules” refer to molecules, which are capable of being dissolved in a solution. In preferred embodiments, the term refers to molecules, which are secreted by or shed from feeder cells. As used herein, the term “soluble molecule expressed by fetal striatal cells” refers both to soluble molecules produced directly by striatal cells, as well as to soluble molecules produced by recombinant means from other cell types (e.g., mammalian, insect, yeast, bacteria, etc.), which have been transfected with a gene typically expressed by fetal striatal cells (e.g., GDNF).

As used herein, the term “recombinant DNA molecule” as used herein refers to a DNA molecule that is comprised of segments of DNA joined together by means of molecular biological techniques. Similarly, the term “recombinant protein” or “recombinant polypeptide” as used herein refers to a protein molecule that is expressed from a recombinant DNA molecule.

The term “adenovirus” as used herein, refers to members of the adenoviridae family of non-enveloped, double-stranded DNA viruses infecting mammals (mastadenovirus) and birds (aviadenovirus). In some embodiments, the present invention utilizes a recombinant adenovirus as a vector for the purpose of expressing the Otx2 transcription factor in ES cells.

As used herein the term “infecting” refers to the process of spreading an infectious agent. In some embodiments, the term “infecting” refers to the process of contacting an ES cell line with recombinant adenovirus particles in a manner which results in the expression of recombinant adenovirus derived nucleic acids in the ES cell line.

The term “positive” as used herein indicates the expression of a gene of interest at a value greater than zero. In preferred embodiments, the term “positive” indicates that the cultured cells express an antigen at a detectable level (e.g., mRNA by RT-PCR, or protein by western blot, immunohistochemistry or flow cytometry) above background (level observed without template, or in the presence of an irrelevant primary antibody or in the presence of the secondary antibody alone).

As used herein, the term “symptoms” refers to the outward manifestations of a disease. For instance, patients with Parkinson's disease exhibit symptoms such as bradykinesia, tremor, rigidity, and/or atypical gait.

The term “alleviating” refers to the act of providing relief from some painful state. As used herein, the term “alleviating” refers to the lessoning of symptoms of Parkinson's disease or to the lessoning of side effects associated with long term L-dopa administration. In some embodiments, the term “alleviating” comprises curing Parkinson's disease. In other embodiments, the term “alleviating: comprises decreasing the severity of the symptoms of Parkinson's disease as measured by any one of several accepted diagnostic measures including but not limited to UPDRS, Schwab and England Scale, and CAPIT.

The terms “Unified Parkinson's Disease Rating Scale” and “UPDRS” refer to a standardized tool used to measure Parkinson's Disease severity, as described by Fahn et al., in Recent Developments in Parkinson's Disease, Fahn et al. (eds.) Plurham Park, N.J.: Macmillian Healthcare Information, 2:153-163, 1987.

“The term “Schwab and England Scale” refers to a standardized tool used to measure the efficacy of surgical treatment for Parkinson's Disease, as described by Schwab and England, in Third Symposium on Parkinson's Disease, Gillingham and Donaldon (eds.) Edinburgh, Scotland: Livingstone, pp. 152-157, 1969.

The terms “Core Assessment Program for Intracerebral Transplantation” and “CAPIT” refer to a standardized tool used to measure the efficacy of cerebral surgery, as described by Langston et al., Mov Disord, 7:2-13, 1992.

Experimental

The following examples are provided in order to demonstrate and further illustrate certain preferred embodiments and aspects of the present invention and are not to be construed as limiting the scope thereof.

In the experimental disclosure which follows, the following abbreviations apply: U (units); N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); L or L (liters); ml (milliliters); μl (microliters); cm (centimeters); mm (millimeters); μm (micrometers); nm (nanometers); ° C. (degrees Centigrade); OD (optical density); pfu (plaque forming units); MOI (multiplicity of infection); bp (base pairs); PCR (polymerase chain reaction); RT (reverse transcriptase); AP (Anterior/Posterior coordinate); VD (Ventral/Dorsal coordinate); LAT (lateral distance coordinate); Ad (adenovirus); BME (β-mercaptoethanol) DAT (dopamine transporter); DMEM (Dulbecco's modified Eagle medium); EDTA (ethylenediaminetetraacetic acid); ES cells (embryonic stem cells); FBS (fetal bovine serum); bFGF (basic fibroblast growth factor); FGF8 (fibroblast growth factor-8); GDNF (glial-derived neurotrophic factor); GFP (green fluorescent protein); GMEM (Glasgow minimal essential medium); HBSS (Hank's Balanced Salt Solution); KSR (knockout replacement serum); LIF (leukemia inhibitory factor); MuLV (murine leukemia virus); NAA (nonessential amino acids); 6-OHDA (6-hydroxydopamine); PBS (phosphate buffered saline); RA (retinoic acid); SDS (sodium dodecyl sulfate); SHH (sonic hedgehog); TH (tyrosine hydroxylase); TP (terminal protein); X-gal (5-bromo-4-chloro-3-indolyl-galactopyranoside); mES (mouse embryonic stem); and hES (human embryonic stem).

Reagents used in the examples below were obtained from: Amersham (Amersham Bioscience, Piscataway, N.J.); BD (BD Biosciences, Discovery Labware, Bedford, Mass.); BresaGen (BresaGen Limited, Athens, Ga.); Calbiochem (Calbiochem-Novabiochem Corporation, San Diego, Calif.); Clontech (BD Biosciences Clontech, Palo Alto, Calif.); Falcon (BD Falcon, Bedford, Mass.); Gibco (Gibco BRL, now Life Technologies); Life Technologies (Invitrogen Life Technologies, Carlsbad, Calif.); MSI (Micron Separation Inc., now Osmonics Inc., Minnetonka, Minn.); NEN (New England Nuclear, now PE); PE (PerkinElmer Life Sciences Inc., Boston, Mass.); Pel-Freeze (Pel-Freez Clinical Systems Inc., Brown Deer, Wis.); Pierce (Pierce Endogen, Rockford, EL); R&D (R&D Systems, Minneapolis, Minn.); Sigma Aldrich (Sigma Chemical Co., St. Louis, Mo.); Specialty Media (Specialty Media, Phillipsburg, N.J.); and Vector Labs (Vector Laboratories, Inc., Burlingame, Calif.).

### EXAMPLE 1

### Construction of Recombinant Adenoviruses

In this Example, recombinant adenoviruses expressing human Otx2 (hOtx2) were constructed as previously described (He et al, Proc. Natl. Acad. Sci. USA, 95:2509-2514, 1998). A plasmid containing the hOtx2 cDNA (obtained from A. Simeone) was digested with BamHI and EcoRI. The resulting hOtx2 fragment was subsequently subcloned into an adenoviral shuttle vector, pACCMVpLpA (obtained from J. Schaack). The sequence of the resulting construct was verified by restriction digestion and DNA sequence analysis. In a similar manner, mouse En2 (mEn2, obtained from A. Joyner) and as a control GFP (Clontech) were subcloned into adenoviral shuttle vectors.

To generate recombinant adenoviruses, shuttle vectors containing hOtx2, mEn2 or GFP were cotransfected with a BstB1-digested Ad5dl327BstBgal-terminal protein (TP) DNA complex into human embryonic kidney cells (293 cells) by calcium phosphate mediated coprecipitation. TP is synthesized as a precursor (pTP), which serves to prime adenovirus replication by covalently binding to the first based of the adenovirus genome. It is later proteolytically processed into mature TP within the virion.

Diluted lysates were used to transfect fresh 293 cells in order to isolate recombinant adenoviruses by plaque purification. In the presence of the x-gal chromogenic substrate, clear plaques were isolated. The plaque-purified viruses were then expanded on a fresh 293 cell monolayer, and analyzed for the presence of hOtx2, mEn2 or GFP by PCR and restriction digestion. Recombinant viruses were plaque purified an additional two times before further expansion. High titer recombinant adenovirus stocks (Ad-hOtx2, Ad-mEn2, and Ad-GFP) were prepared by purification of the viruses on consecutive step and isopycnic cesium chloride gradients. Viruses were dialyzed for 1 hr each against three changes of 10 mM Tris-HCl, pH 7.9, 135 mM NaCl, 1 mM MgCl₂, 50% v/v glycerol. Purified virus stocks were stored at −20° C. prior to use. Viral particle concentrations were determined spectrophotometrically, by measuring the absorbance at 260 nm, where 1.0 units at A₂₆₀ was estimated to contain 10¹² particles. Plaque titers were determined using 293 cells. In all cases, the particle-to-plaque forming unit ratio was close to 100%.

### EXAMPLE 2

### Culture of Undifferentiated Mouse and Human ES Cells

Briefly, undifferentiated murine ES cells (mES, obtained from G. Keller) were grown in mouse ES cell medium on gelatin-coated 100 mm tissue culture plates (BD). Before use, the tissue culture plates were coated with 0.1% gelatin in PBS for 20 min at room temperature, followed by three washes with Hanks balanced buffer. The recipe for mouse ES cell medium was: DMEM with high glucose, with L-glutamine, without pyruvate, 10% ES-grade fetal bovine serum, 0.1 mM MEM nonessential amino acids (NAA), 0.1 mM β-mercaptoethanol (BME, Sigma), 100 U/ml recombinant human leukemia inhibitory factor (LIF, Chemicon), and 100 U/ml penicillin/streptomycin. Unless otherwise indicated, all reagents were obtained from Gibco. When grown to confluence, ES cells were passaged by dissociation with 0.05% trypsin/0.04% EDTA (Sigma), diluted and then plated on fresh gelatin-coated plates at four day intervals.

Human ES cells (hES of the BGU#1 line generously provided by BresaGen Limited) were maintained in an undifferentiated state on fetal mouse fibroblast feeder cell layers (Specialty Media) in human ES cell medium. The recipe for human ES cell medium was: DMEM/F12 1:1, containing 15% fetal bovine serum, 5% knock-out serum replacement (KSR, Gibco), 100 units/ml LIF, 0.1 mM BME, 2 mM L-glutamine, 1% penicillin/streptomycin, 0.1 mM NAA, and 4 ng/ml recombinant human basic fibroblast growth factor (bFGF, Sigma). hES cells were manually dissected and plated for subsequent passages and for in vitro differentiation.

### EXAMPLE 3

### Differentiation of Mouse and Human ES Cells

In this example, multiple methods for differentiation of mouse and human ES cells into TH positive neurons are described. The present invention is not limited to any single embodiment, and in fact modifications of these techniques are contemplated to be within the scope of the present invention.

Differentiation of mES Cells with Methods Comprising RA, SHH and FGF8

On day 0, undifferentiated murine ES cells were dissociated with trypsin/EDTA and seeded on gelatin-coated 6-well tissue culture plates (Costar), in 2 ml growth medium at a concentration of 1×10⁵ cells/ml. The following day, the ES cells were infected with either Ad-hOtx2 or Ad-mEn2 at an MOI of 200. On day 2, the adenovirus-containing medium was removed and replaced with fresh ES cell growth medium. On day 4, the ES cells were dissociated with trypsin/EDTA and plated on gelatin-coated 6-well plates in 2 ml ES cell differentiation medium. The recipe for the ES cell differentiation medium was: DMEM with high glucose, with L-glutamine, without pyruvate, 15% ES cell grade FBS, 0.1 mM MEM nonessential amino acids, 0.1 mM 2-mercaptoethanol (Sigma), and 100 U/ml penicillin/streptomycin. Unless otherwise indicated, all reagents were obtained from Gibco. Retinoic acid (RA) obtained from Sigma, was added to the culture medium to initiate differentiation at a final concentration of 1 μM. The dopamine neuron inducing factors Sonic hedgehog (SHH) and fibroblast growth factor-8 (FGF8), both obtained from R&D, were also added at this time at final concentrations of 100 ng/ml and 50 ng/ml, respectively. On day 6, the spent ES cell differentiation medium was replaced with fresh ES cell differentiation medium containing RA, SHH and FGF8 at the same final concentrations as used on day 4. On day 6, the differentiated ES cells were harvested for analysis by RT-PCR, western blotting and immunocytochemistry, as described in the following Examples.

Differentiation of mES Cells with Methods Comprising SHH, FGF8, and N-2

Another technique was also used successfully to differentiate murine ES cells. Briefly, on day 0 undifferentiated ES cells were seeded on gelatin-coated 6-well plates at a density of 20,000 cells/cm² in DMEM (high glucose, with glutamine), 10% ES cell-grade FBS, 0.1 mM MEM nonessential amino acids, and 100 U/ml penicillin/streptomycin. The following day, half of the culture medium volume was removed, and the ES cells were infected with Ad-hOtx2 at an MOI of 200 pfu/cell. The plates were gently rocked hourly 3-4 times. After 4 hrs, fresh culture medium was added to the wells to bring the volume back to the starting volume. On day 2, all of the adenovirus-containing medium was removed and replaced with fresh culture medium. To this medium, FGF-8 was added to a final concentration of 100 ng/ml. On day 4, the spent medium was removed and replaced with fresh culture medium. Both SHH and FGF-8 were added to the medium to a final concentration of 100 ng/ml each. On day 6, the cells were further differentiated by replacing the spent medium with N-2 medium and incubating for another 3 days. N-2 medium consists of: DMEM/F12 1:1, containing 1× N2, 1% FBS, 2 mM glutamine, 100 U/ml penicillin/streptomycin, 100 ng/ml SHH, and 100 ng/ml FGF8. Alternatively, the cells can be further differentiated, by coculturing them with rat embryonic striatum cells. The day 15 fetal rat striatum cells were first mechanically dissociated and plated in polyethyleneimine-coated plates at 20,000 cells/cm² in culture medium composed of Ham's F12 medium (Irvine Scientific) containing 5% human placental serum, 2 mM glutamine, and 100 U/ml penicillin/streptomycin. The day 7 ES cells were seeded on the rat striatum cultures at 10,000 cells/cm², and cultured for an additional 3-5 days.

Differentiation of mES Cells with Methods Comprising PA6 Cell and Fetal Striatum

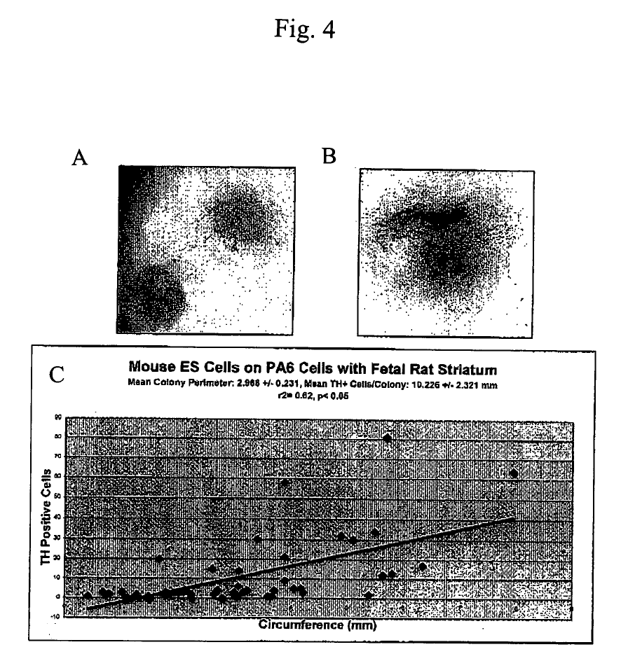

Briefly, mES cells were cultured on PA6 cells for one week in a serum-free differentiation medium: Glasgow Minimal Essential Medium (GMEM, Gibco) containing L-glutamine, 10% KSR, 0.1 mM BME, 1% sodium pyruvate, 1% penicillin/streptomycin, and 0.1 mM NAA. Fragments of fetal rat striatum obtained from embryonic day 14 rats were then added to cultures (e.g., approximately 0.6 fetal striatum/cm² tissue culture plate). After incubation of the mES cells in the presence of rat striatum for one week, the cells were fixed with 4% paraformaldehyde and processed for immunohistochemistry as described in Example 6 below. As shown in FIG. 4, TH positive cells were detected in mES cell colonies differentiated by this method. Panel C depicts a positive correlation between differentiated mES cell colony circumference and TH positive cell number.

Differentiation of hES Cells with Methods Comprising PA6 Cells and Fetal Striatum

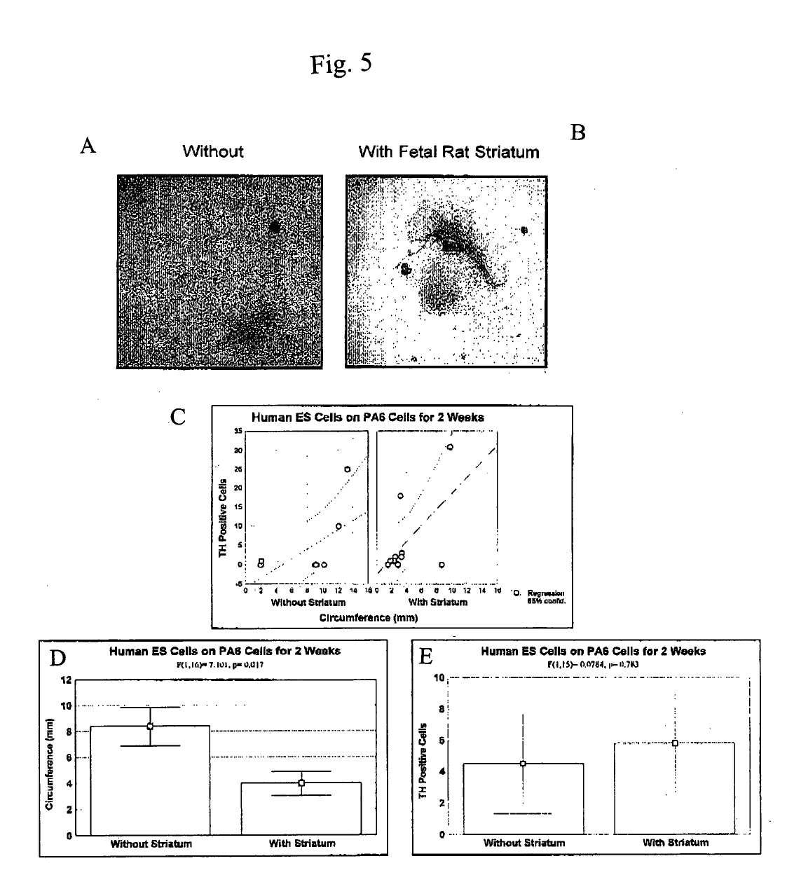

Importantly, the method described above for differentiation of mES cells on PA6 cells was also used successfully to obtain TH positive cells from undifferentiated hES cells. Briefly, hES cells were cultured for one week with PA6 cells in serum-free differentiation medium (GMEM containing L-glutamine, 10% KSR, 0.1 mM BME, 1% sodium pyruvate, 1% penicillin/streptomycin, and 0.1 mM NAA). The hES cells were cultured for a second week in the presence or absence of fetal rat striatum fragments, and then fixed for immunohistochemistry with 4% paraformaldehyde. As shown in FIG. 5, small numbers of TH positive cells were detected in hES cell colonies differentiated by this method regardless of whether rat striatum fragments were added to the cultures.

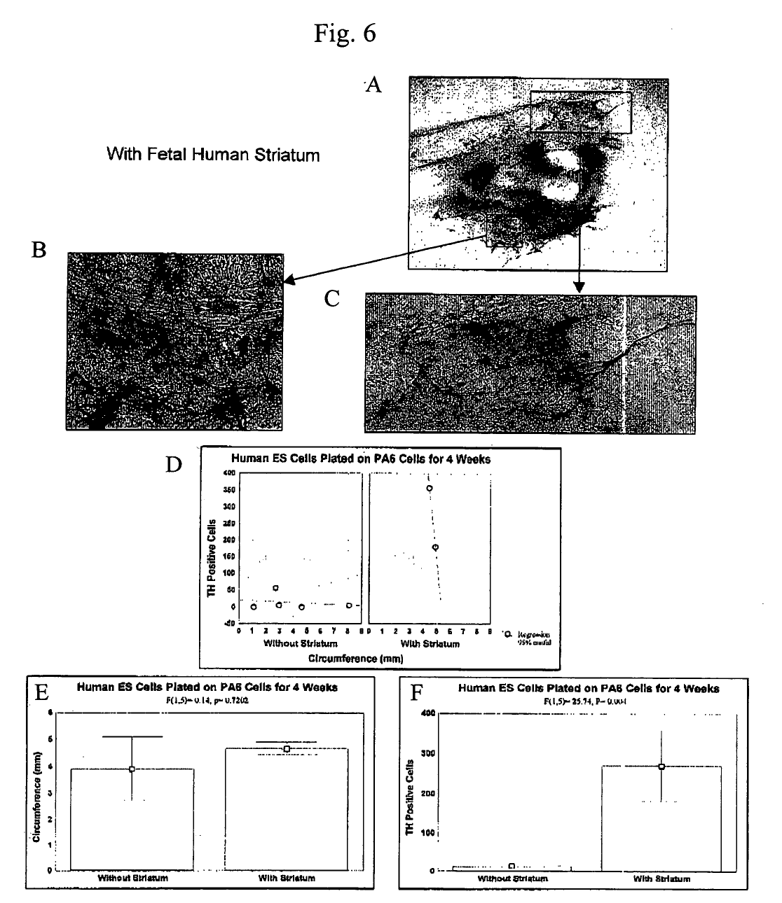

In a further series of experiments conducted during development of the present invention, hES cells were cultured overnight on PA6 cells in 24 well plates. The tissue culture medium was replaced with serum-free differentiation medium (GMEM containing L-glutamine, 10% KSR, 0.1 mM BME, 1% sodium pyruvate, 1% penicillin/streptomycin, and 0.1 mM NAA) and fragments of human striatum obtained from 8 week old fetuses were placed in contact with the cultures using cell culture inserts (Falcon) with a semipermeable membrane. The Falcon tissue culture inserts used had pores of about 4 microns, permitting diffusion of molecules secreted by the fetal striatal cells without ES cell/striatal cell contact. After 4 4 weeks in culture, the inserts were removed and the hES cells were fixed with 4% paraformaldehyde for microscopy. As shown in FIG. 6, coculturing hES cells on PA6 cells in the presence of fetal human striatum for four weeks, yielded greater numbers of TH positive cells than did methods lacking striatal cells.

Differentiation of hES Cells with Methods Comprising Fetal Astrocytes

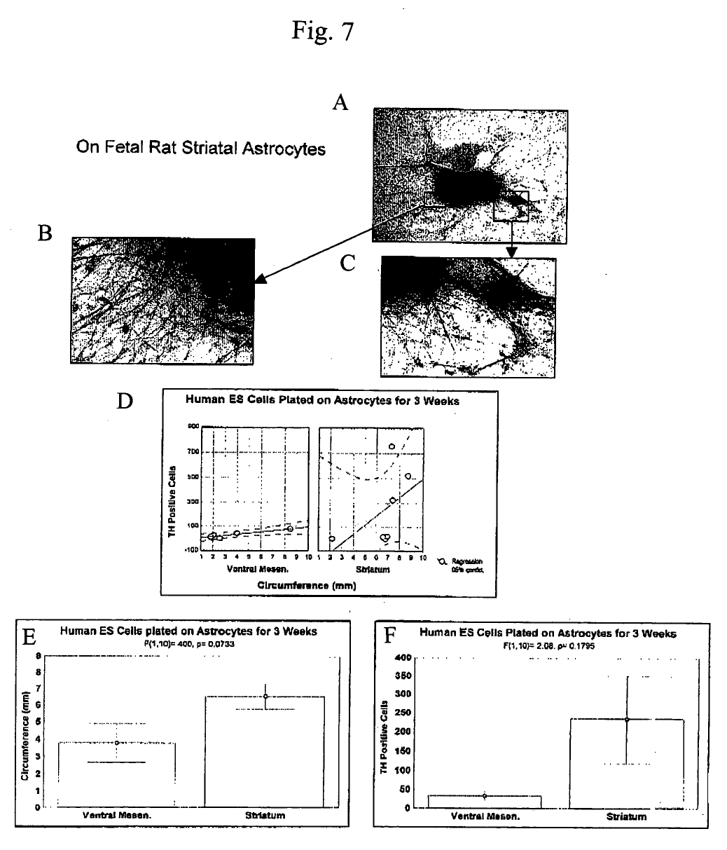

In these experiments, fragments of fetal rat striatum or fetal rat ventral mesencephalon were repeatedly pipetted using a 1 ml pipette tip to semi-detach the cells. The fetal rat tissues were cultured to allow astrocyte attachment, and then neurons, as well as other unattached cells, were dislodged by shaking the plates overnight permitting their removal by aspiration. hES cells were added to plates containing the striatal and ventral mesencephalon astrocytes in serum-free differentiation medium (GMEM containing L-glutamine, 10% KSR, 0.1 mM BME, 1% sodium pyruvate, 1% penicillin/streptomycin, and 0.1 mM NAA). The cells were cultured for three weeks and then fixed with 4% paraformaldehyde for immunohistochemistry. As shown in FIG. 7, hES cells plated on striatal astrocytes for three weeks yielded larger hES cell colonies containing greater numbers of TH cells.

Taken together, the results obtained during development of the present invention indicate that soluble factors derived from fetal striatal tissue enhance differentiation of ES cells to a TH positive, dopamine neuron phenotype. However, an understanding of any of the mechanism(s) involved is not necessary in order to make or use the present invention, and it is not intended that the present invention be limited to any particular mechanism(s).

### EXAMPLE 4

### RT-PCR Analysis of Differentiated ES Cells

The mouse and human ES cells differentiated as described in Example 3, were washed once with cold sterile PBS. One ml Trizol reagent (Life Technologies) was added to each well of the 6 well plates to lyse the ES cells. The lysates were collected into 1.5 ml tubes and homogenized by passage through a 1 ml pipette tip 5-10 times. The samples were then incubated for 5 min at room temperature, prior to the addition of 0.2 ml chloroform per 1 ml Trizol. After centrifugation at 13,000 rpm for 15 min, the supernatant was collected. RNA was precipitated from the supernatant by adding 0.5 ml isopropyl alcohol and centrifugation at 13,000 rpm for 10 min. The RNA pellet was washed once with 75% ethanol, air dried and then dissolved in RNase-free water. The RNA concentration was determined by measuring the OD_{260/280}.

To generate first strand cDNA, 1 μg of DNase I-digested total RNA was reverse transcribed using random hexamer primers and MuLV reverse transcriptase (Perkin Elmer). The 20 μl reaction mixture consisted of: 1 μg RNA in 3 μl water, 1 μl hexamer primers, 2 μl 10× PCR buffer II, 4 μl 25 mM MgCl₂, 8 μl 2.5 mM dNTP, 1 μl RNase inhibitor, 1 μl MuLV reverse transcriptase. The reaction mixture was incubated at 42° C. for 15 min, followed by a denaturation step at 99° C. for 5 min, and a 4° C. cooling step.

The following oligonucleotide primer pairs were used for detection of dopamine neuron antigen expression: Tyrosine hydroxylase (TH) sense primer 5′-GTT CTC CCA GGA CAT TGG AC-3′ (SEQ ID NO:1) and TH antisense primer 5′-GCT GGA TAC GAG AGG CAT AG-3′ (SEQ ID NO:2) predicted to yield a 320 bp PCR product; dopamine transporter (DAT) sense primer 5′-AAC TCC CTG ACG AGC TTC TC-3′ (SEQ ID NO:3) and DAT antisense primer 5′-CAT GGC ACT GTC GAT ACC CA-3′ (SEQ ID NO:4) predicted to yield a 215 bp PCR product; Nurr1 sense primer 5′-TTG TGT TCA GGC GCA GTA TG-3′ (SEQ ID NO:5) and Nurrl1 antisense primer 5′-GCA TCT GAA TGT CTT CTA CC-3′ (SEQ ID NO:6) predicted to yield a 260 bp PCR product; En2 sense primer 5′-AGA GGC TCA AGG CTG AGT TTC-3′ (SEQ ID NO:7) and En2 antisense primer 5′-CGC TTG TTC TGG AAC CAA ATC-3′ (SEQ ID NO:8) predicted to yield a 130 bp PCR product; and Otx2 sense primer 5′-CAC CTC CAA ACA ACC TTA GC-3′ (SEQ ID NO:9) and Otx2 antisense primer 5′-GGT TCT ACA GGT CTT CAT CA (SEQ ID NO:10) predicted to yield a 410 bp PCR product.

Each PCR reaction consisted of: 34.2 μl water, 4.5 μl 10× PCR buffer II, 3 μl 25 mM MgCl₂, 2 μl dNTPs (2.5 mM), 0.5 μl sense primer, 0.5 μl antisense primer, 0.3 μl Taq DNA polymerase (5 U/μl) and 5 μl first-strand cDNA. The PCR conditions were optimized for each gene. Samples were denatured at 94° C. for 2 min, followed by 25-30 cycles of PCR consisting of a 94° C. denaturation step for 30 sec, a 55-60° C. annealing step for 30 sec, and a 72° C. extension step for 30 sec. After the final cycle, a 72° C. extension step for 10 min was done. The PCR products were examined by agarose gel electrophoresis.

### EXAMPLE 5

### Western Blot Analysis of Differentiated ES Cells

The mouse and human ES cells differentiated as described in Example 3, were washed once with cold sterile PBS. The ES cells were then harvested in lysis buffer (10 mM Tris-HCl pH 7.8, 10 mM NaCl, 0.5% NP-40, 0.1% SDS, 1 μM PMSF and 1 μg/ml aprotinin, all from Sigma). After homogenization, the lysates were centrifuged at 13,000 rpm for 15 min to remove debris. The protein concentration of the lysates was determined by the BCA method (Pierce). Approximately 20 μg/lane protein was separated on a 12% polyacrylamide gel and transferred to a nitrocellulose membrane (MSI). The nitrocellulose was blocked for 30 min in 5% dry milk in PBS containing 0.3% Tween-20, followed by incubation overnight at room temperature with either 1:5,000 rabbit anti-TH (Pel-Freeze) or 1:20,000 dilution of a polyclonal rabbit anti-DAT serum (obtained from the National Institute of Drug Abuse). After washing, the blot was incubated for 1 hr in a solution containing a polyclonal peroxidase-conjugated goat anti-rabbit antibody (Vector) and developed using chemiluminescent reagents (NEN).

### EXAMPLE 6

### Immunohistochemistry of Differentiated ES Cells

The mouse and human ES cells differentiated as described in Example 3, were washed once with cold sterile PBS. The cells were fixed with 4% paraformaldehyde for 30 min followed by three PBS washes. After washing, the cells were incubated in blocking buffer (5% normal goat serum, 0.1% Triton X-100 in PBS) for 30 min. The ES cells were stained with primary antibodies overnight: 1:500 rabbit anti-TH (Pel-Freeze); and 1:200 rabbit anti-DAT. The cells were then washed with PBS and incubated with 1:200 goat anti-rabbit IgG (Vector) for 2 hr. After washing, the cells were incubated with 1:100 ABC reagent (Vector) for 1 hr, and developed with diaminobenzidine (DAB, obtained from Sigma). For dual immunofluorescence analysis, α-synuclein was imaged with a 1:50 dilution of a Texas Red antibody (Amersham), and TH was imaged with a 1:40 dilution of a FITC antibody (Calbiochem).

### EXAMPLE 7

### Implantation of Cultured Dopamine Neurons in Hemiparkinsonian Rats

The survival of transplanted dopamine neurons generated in vitro from ES cells using the methods described above in Example 3, are described. For these studies, rats with unilateral dopamine lesions are used as transplant recipients (Richards et al., Pharmacol. Biochem. Behav., 36:217-223, 1990).

Lesioning rat median forebrain bundle: For lesioning, 20 μg of 6-OHDA are injected at two sites, AP: −2.1 mm posterior to bregma, LAT: 2.0 mm from the midline, VD: −7.8 mm below the dura; and AP: −4.3 mm posterior to bregma, LAT: 1.5 mm from the midline, VD: −7.9 mm below the dura. Once the rats successfully recover from the lesioning procedure (7 to 10 days post 6-OHDA injection), the animals are tested to assess the extent of the striatal lesion using a methamphetamine-induced rotation (circling) behavioral test. Rats that do not show a positive result in the behavioral test are eliminated from subsequent analysis. Only animals circling above 3 rpm in ipsilateral direction are used. After lesioning, the animals are grouped such that each group mean circling rate is around 8 rpm. Animals showing successful lesioning are tested again at 3 weeks and again at 6 weeks.

Transplantation: Two to six weeks are allowed to pass between lesioning and transplantation. For the transplantation, the in vitro-differentiated dopamine neurons (See, Example 3) are suspended by trypsinization and washed extensively in Ca²⁺/Mg²⁺ free Hank's Balanced Salt Solution (HBSS). The dopamine neurons are transplanted into the denervated rat striatum by drawing the neurons up into a 24 g sterile stainless steel cannula attached to a microliter syringe and stereotaxically transplanted at the following coordinates (AP 0.0 mm form bregma, LAT3.0 mm form the midline, VD−3.5 to −7.5 mm below the dura) in 4.0 μl over 4 min.

Visualization of TH+ cells: Six weeks after transplantation, all animals are sacrificed, and their brains are perfused with heparinized-saline followed by 4% paraformaldehyde. The brains are sectioned on a cryotome at 40 micrometer thickness and immunostained for tyrosine hydroxylase (TH) using a polyclonal anti-rat TH antibody and a peroxidase-based ABC kit. All TH-immunoreactive neurons are counted in every third section under a microscope. Abercrombie's correction is used to estimate the total number of TH+ neurons surviving in each transplant.

### EXAMPLE 8

### Effects of Transplanted Fetal Neuronal Tissue on Methamphetamine-Induced Rotation

In this Example, experiments to determine the effects of transplanted in vitro-differentiated dopamine neurons on methamphetamine-induced rotation are described.

Lesioned rat striatum: Rat substantia nigra dopamine neurons are lesioned with 6-OHDA, as described in the previous Example.

Transplantation: Rats receive in vitro-differentiated dopamine neurons transplants, as described above. In addition, a group of rats receive a sham transplantation consisting of buffer vehicle only.

Methamphetamine treatment: At three weeks and six weeks following the transplantation, rats are injected i.p. with 5.0 mg/kg methamphetamine. Methamphetamine-injected animals are placed in vertically positioned plexiglass drums and attached to a flexible cable that records rotations to a computer. Animals are tested for 120 minutes. During the initial 30 minutes of testing, the effects of methamphetamine develop and rotations ipsilateral to the lesion are recorded in each animal every 10 minutes during the subsequent 90 minute interval. The average ipsilateral rotations per minute (rpm) are then calculated for each animal.

Results: Transplants of in vitro-differentiated dopamine neurons are contemplated to accelerate the rate of behavioral recovery when compared to the sham buffer-alone transplants.

### EXAMPLE 9

### Implantation of Cultured Dopamine Neurons in Human Parkinson's Patients

Briefly, hES cells differentiated in vitro are transplanted into the skulls of human Parkinson's patients as previously described (Freed et al., N. Engl. J. Med., 344:710-719, 2001). Differentiated hES cells are harvested manually from dopamine producing cultures (determined by measuring homovanillic acid concentrations in culture medium) and deposited into the patient's putamen. The patient is given only local anesthesia in order to monitor the patient's ability to speak and move their extremities after each injection. The transplant efficacy is measured by ¹⁸F-fluorodopa PET scanning, and by analysis of gross clinical improvement or deterioration.

Additional compositions and methods suitable for transplantation of cultured dopamine neurons and assessment of transplant affect on the cognitive function of Parkinson's patients are known in the art (Trott et al., Neurology, 60:1938-1943, 2003, herein incorporated by reference).

### EXAMPLE 10

### Optimization of Dopamine Neuron Production

Briefly, green fluorescent protein (GFP) is used as a reporter gene to monitor embryonic stem cell differentiation. In some embodiments, GFP (by itself or as a fusion protein) expression is placed under control of the human dopamine transporter gene promoter, in hES cell transfectants. Upon differentiation of the hES cell transfectants into dopamine neurons, GFP is expressed, permitting the selection of pure populations of in vitro-differentiated dopamine neurons via fluorescent activated cell sorting. To isolate cells earlier in the differentiation process, hES cells are transfected with plasmids containing GFP driven by other promoters (e.g., nestin, aldehyde-dehydrogenase-1, engrailed, etc.).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the relevant fields, are intended to be within the scope of the following claims.

We claim:

1. A method for producing tyrosine hydroxylase-positive neurons, comprising the steps of:
a) providing a human embryonic stem cell line; and
b) contacting said embryonic stem cell line with a solution comprising at least one soluble molecule expressed by fetal striatal cells, under conditions suitable for producing tyrosine hydroxylase-positive neurons.

2. The method of claim 1, wherein said fetal striatal cells are astrocytes

3. The method of claim 1, wherein said fetal striatal cells are cocultured with said human embryonic stem cell line.

4. The method of claim 1, wherein said fetal striatal cells are separated from said human embryonic stem cell line by a semipermeable membrane.

5. The method of claim 1, wherein said at least one soluble molecule comprises glial-derived neurotrophic factor.

6. The method of claim 1, wherein said at least one soluble molecule is provided by conditioned medium from fetal striatal cell cultures.

7. The method of claim 6, wherein said conditioned medium is free of said fetal striatal cells.

8. The method of claim 1, further comprising contacting said human embryonic stem cell line with stromal cells.

9. The method of claim 1, further comprising step c, enriching said tyrosine hydroxylase positive neurons.

10. The method of claim 9, wherein said tyrosine hydroxylase positive neurons are enriched, by selecting colonies with a circumference greater than 4 mm.

11. A cell culture produced by a method comprising contacting a human embryonic stem cell line with at least one soluble molecule expressed by fetal striatal lo cells, under conditions suitable for producing tyrosine hydroxylase-positive neurons.

12. The culture of claim 11, wherein said method further comprises enriching said tyrosine hydroxylase positive neurons.

13. The culture of claim 11, wherein said at least one soluble molecule comprises glial-derived neurotrophic factor.

14. The culture of claim 11, wherein said neurons are suitable graft material for human transplantation.

15. The culture of claim 14, wherein said human transplantation comprises treatment of a subject displaying symptoms of Parkinson's disease.

16. A method of alleviating Parkinson's disease symptoms in a patient with Parkinson's disease comprising:
a) providing a cell culture produced by a method comprising contacting a human embryonic stem cell line with at least one soluble molecule expressed by fetal striatal cells, under conditions suitable for producing tyrosine hydroxylase-positive neurons; and
b) administering said cultured cells comprising tyrosine hydroxylase-positive neurons to the putamen of a patient with Parkinson's disease, under lo conditions suitable for alleviating Parkinson's disease symptoms.

17. The method of claim 16, wherein said at least one soluble molecule comprises glial-derived neurotrophic factor.

18. The method of claim 16, wherein said alleviating Parkinson's disease symptoms is assessed by a technique chosen from the Unified Parkinson's Disease Rating Scale, the Schwab and England Scale, and the Core Assessment Program for Intracerebral Transplantation.

19. The method of claim 18, wherein said patient has advanced Parkinson's disease.

20. A composition comprising at least one human embryonic stem cell and medium comprising glial-derived neurotrophic factor.

21. The composition of claim 20, wherein said glial-derived neurotrophic factor is a recombinant protein.

22. The composition of claim 20, wherein said medium further comprises at least one soluble molecule expressed by stromal cells.
